(19) United States
(12) Patent Application Publication
(10) Pub. No.: US 2013/0337052 A1
(43) Pub. Date: Dec. 19, 2013

(54) System and Method of Reducing Impairment of Alertness, Concentration, Motivation, and Creativity Caused by Medication

(71) Applicants: Patricia Linert, Alameda, CA (US); Kenneth J. Ouimet, Davis, CA (US)

(72) Inventors: Patricia Linert, Alameda, CA (US); Kenneth J. Ouimet, Davis, CA (US)

(21) Appl. No.: 13/625,783

(22) Filed: Sep. 24, 2012

Related U.S. Application Data
(60) Provisional application No. 61/661,724, filed on Jun. 19, 2012.

(51) Int. Cl. A61K 33/00; A61K 33/14; A61K 31/513; A61K 31/198; C01D 15/08; C07C 59/265; C01D 15/04; C07D 239/557; C07C 229/24; A61K 31/232; A61K 31/352; A61K 9/48; A61P 25/20; A61P 25/26; A61K 31/194

(52) U.S. Cl. 424/451; 424/715; 514/574; 424/677; 514/274; 514/561; 423/421; 562/584; 423/499.3; 544/314; 562/571; 514/549; 514/454

(57) ABSTRACT

Administering a therapeutically effective dose of lithium ions mitigates the side effects of a psychoactive substance such as a cannabinoid. The therapeutically effective dose of lithium ions includes greater than 4 milligrams of lithium and less than 170 milligrams of lithium, or includes between 8 and 32 milligrams of lithium ions per milligram of the psychoactive substance. The therapeutically effective dose of lithium ions is administered using lithium carbonate, lithium citrate, lithium chloride, lithium orotate, lithium aspartate, or analogs thereof using a delivery vehicle selected from pills, tablets, capsules, gelcaps, liquids, syrups, injectable liquids, powders, or foods and administered prior to, with, or after administration of the psychoactive substance. The psychoactive substance includes one or more of anandamide, 2-arachidonoyl glycerol, 2-arachidonoyl glycerol ether, tetrahydrocannabinol, cannabinol, cannabidiol, or analogs thereof and may be administered using the delivery vehicle.

### CLAIM TO DOMESTIC PRIORITY

The present application claims the benefit of U.S. Provisional Application No. 61/661,724, filed Jun. 19, 2012, which application is incorporated herein by reference.

### FIELD OF THE INVENTION

The present invention relates in general to a method for increasing a person's alertness, ability to concentrate, and creativity, and more specifically, to a method and composition for increasing the alertness, concentration, motivation, and creativity of a person using medications such as, e.g., Marinol®, through the administration of lithium compounds.

### BACKGROUND OF THE INVENTION

Mammals such as human beings are comprised of billions of individual cells. Each cell has a cell membrane that surrounds the cell like a skin. The cell membrane separates and protects the interior of cells from the external environment around the cell. However, the cell membrane includes molecules and structures that selectively permit materials and information to pass in to and out of the cell. In particular, cell membranes contain structures known as receptors that react to a stimulus outside the cell to provoke a response inside the cell. Receptors are essential to the operation and survival of cells, but the exact manner by which many receptors operate is not fully understood. There are a number of different types of cell membrane receptors.

Stimuli that cause a receptor to provoke a response within the cell are called receptor agonists, and stimuli that inhibit the ability of a receptor to provoke a response within the cell are called receptor antagonists. Each type of receptor typically has a different collection of agonists and antagonists, and a particular stimulus, such as a particular chemical, can be an agonist for more than one receptor and simultaneously an antagonist for one or more other receptors. Furthermore, some receptors can provoke one or more of a number of different responses within the cell depending on which particular agonist is stimulating the receptor and the conditions present in and around the cell at the time. Accordingly, predicting how different receptor agonists and antagonists, e.g., different medications, will interact with each other is difficult. Predicting how an agonist or antagonist will interact with a change in the conditions in and around the cell, such as, for example, a change in the concentration of a biologically active ion, is also difficult.

Many medications act by interacting with a cell membrane receptors, either by stimulating the receptor, i.e., by being a receptor agonist, or by preventing the receptor from being stimulated, i.e., by being a receptor antagonist. For example, losartan and some other medications used to treat high blood pressure are angiotensin II receptor antagonists. Angiotensin II receptors operate to provoke vasoconstriction (narrowing of blood vessels) and increased retention of water, thereby increasing blood pressure. Blocking the operation of angiotensin II receptors with an antagonist such as losartan reduces blood pressure. Other medications that are receptor agonists or antagonist include the anti-retroviral medication maraviroc, the anti-inflammatory medication epinephrine, and the anti-cancer medication tamoxifen.

However, because receptor agonists and antagonists often interact with more than one receptor and each receptor is often involved in more than one cellular process, receptor agonist and antagonist medications often have undesirable side effects. For example, losartan can cause miscarriages, epinephrine can cause irregular heartbeat, and tamoxifen can cause memory impairment. Many promising receptor agonist or antagonist medications failed to gain regulatory approval or were withdrawn from the market because of the discovery of potentially dangerous side effects.

One type of cell membrane receptor being studied for therapeutic use is cannabinoid receptors. In mammals including human beings, cannabinoid receptors are found primarily in cells of the nervous and immune systems. Two distinct types of cannabinoid receptor, types 1 (CB₁) and 2 (CB₂), have been identified. CB₁ receptors are commonly found in brain cells, and CB₂ receptors are commonly found in immune system cells. Cannabinoid receptors react to naturally occurring chemicals in the body known as endocannabinoids, such as arachidonoyl ethanolamide (anandamine) and 2-arachidonoyl glycerol (2-AG). While the methods of operation and biochemical roles of endocannabinoids are not fully understood, research indicates that endocannabinoids are involved in a variety of normal bodily functions such as controlling appetite, sensing pain, creating and maintaining memories, responding to stress, and reducing inflammation, among other functions. Cannabinoid receptors also react to chemicals from outside the body, accordingly known as cannabinoids. As used herein, the term cannabinoid includes all cannabinoid receptors agonists and antagonists including endocannabinoids.

Cannabinoid receptors are a key biochemical pathway by which some prescription medications operate. For example, the cannabinoid Marinol® (generic name dronabinol, active ingredient (−)-trans-Δ9-tetrahydrocannabinol) is currently approved by the U.S. Food and Drug Administration (FDA) for treating anorexia in AIDS patients and nausea and vomiting in chemotherapy patients. The therapeutic effects of Marinol arise from Marinol operating as a cannabinoid receptor agonist. Marinol and other synthetic and natural substances that act as cannabinoid receptor agonists or antagonists are used or being investigated for use in a variety of therapeutic roles, including for treatment of pain, glaucoma, inflammatory diseases, osteoporosis, atherosclerosis, Alzheimer's disease, strokes, and brain tumors.

Marinol and other cannabinoid receptor agonists and antagonists have side effects such as, e.g., drowsiness, inability to concentrate, decreased motivation, and decreased creativity. These side effects can impair the ability of patients to perform ordinary activities. For example, a person using a cannabinoid medication such as Marinol may be unable to maintain employment because the medication prevents the person from concentrating adequately on the tasks they are required to perform or causes the person to doze off at inappropriate times. A person using a medication such as Marinol may also be unable or lack the motivation needed to perform basic grooming and housekeeping tasks. In such cases, the side effects of the medication may be as detrimental to the patient as the original disease or injury that the medication is intended to treat. The patient confronts a choice between two undesirable options: forego treatment and suffer the effects of the injury or disease, or undergo treatment and suffer the debilitating side effects of the medication.

### SUMMARY OF THE INVENTION

A need exists to reduce the impairment of alertness, concentration, motivation, and creativity caused by cannabinoid medications. Accordingly, in one embodiment, the present invention is a method of mitigating side effects of a cannabinoid comprising the step of administering a therapeutically effective dose of lithium ions.

In another embodiment, the present invention is a pharmaceutical composition for mitigating side effects of a cannabinoid comprising a therapeutically effective dose of lithium ions.

In another embodiment, the present invention is a method of making a pharmaceutical composition for mitigating side effects of a cannabinoid comprising the steps of providing a lithium compound including a therapeutically effective dose of lithium ions, providing a cannabinoid, and incorporating the lithium compound and the cannabinoid into a delivery vehicle.

In another embodiment, the present invention is a method of making a pharmaceutical composition for mitigating side effects of a cannabinoid comprising the steps of providing a lithium compound including a therapeutically effective dose of lithium ions and incorporating the lithium compound into a delivery vehicle.

### BRIEF DESCRIPTION OF THE DRAWINGS

FIGS. 1A-1F show the chemical structure of chemicals that have or affect activity of the CB₂ and/or CB₂ receptors;

FIGS. 2A-2D show the chemical structure of therapeutic lithium compounds;

FIG. 3 shows a method of treating a human with lithium compounds;

FIGS. 4A-4H show delivery vehicles for administering lithium compounds and for administering combinations of cannabinoids and lithium compounds;

FIGS. 5A-5B show a method of making a gelcap including a lithium compound or including a combination of a cannabinoid and a lithium compound;

FIGS. 6A-6C show a method of making a pill including a lithium compound or including a combination of a cannabinoid and a lithium compound; and

FIGS. 7A-7D show a method of making a food including a lithium compound or including a combination of a cannabinoid and a lithium compound.

### DETAILED DESCRIPTION OF THE INVENTION

The present invention is described in one or more embodiments in the following description with reference to the figures, in which like numerals represent the same or similar elements. While the invention is described in terms of the best mode for achieving the invention's objectives, it will be appreciated by those skilled in the art that it is intended to cover alternatives, modifications, and equivalents as may be included within the spirit and scope of the invention as defined by the appended claims and their equivalents as supported by the following disclosure and drawings.

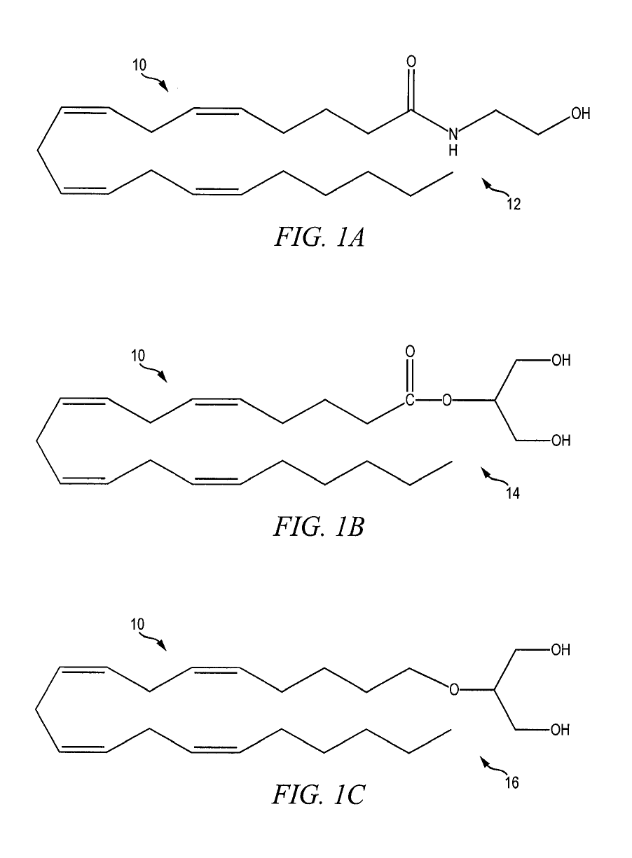

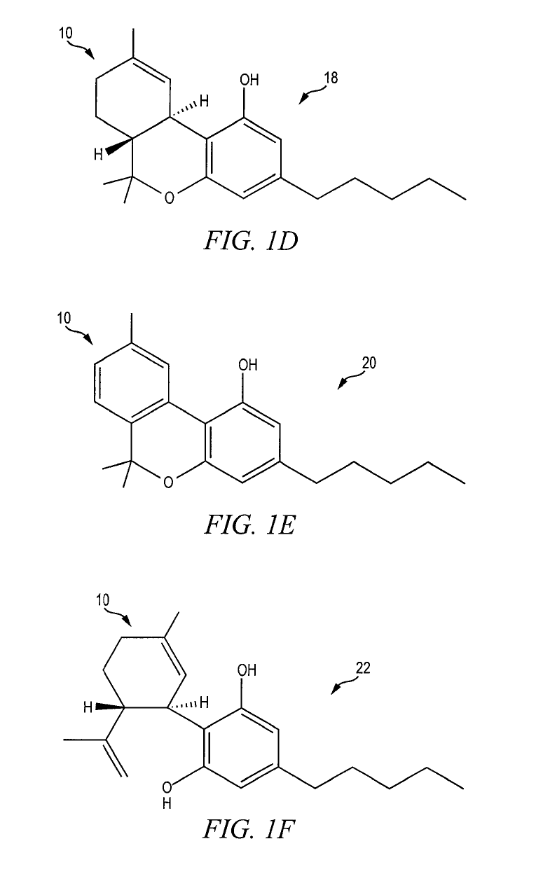

FIGS. 1A-1F show the chemical structure of substances known to act as agonists and/or antagonists of the cannabinoid receptors CB₂ and CB₂, which substances are generally referred to as cannabinoids 10. The discovery of cannabinoid receptors in human cells in the early 1990s triggered research into the roles of the receptors in the body and led to the discovery of the naturally occurring endocannabinoids that work with the receptors. As the many roles that cannabinoid receptors and endocannabinoids played emerged, researchers sought to develop new medications and therapies that would exploit the operation of the cannabinoid receptors to treat physical and psychiatric problems such as chronic nausea, anorexia, obesity, glaucoma, depression, and chronic pain. The research has revealed the diversity and complexity of the internal biochemical processes that create, employ, and break down cannabinoids.

Because the cannabinoid receptors are involved in a large number of cellular processes, and because substances that act on the cannabinoid receptors sometimes act in other ways as well, the search for successful cannabinoid-receptor-based therapies has been difficult and unpredictable. For example, the cannabinoid rimonabant is a CB₁ antagonist that was approved for the treatment of obesity. Subsequently, researchers came to believe that rimonabant had triggered severe depression and suicidal thoughts in many patients. Rimonabant was subsequently withdrawn from the market.

In contrast, other cannabinoids have proven to be effective therapies for a number of serious ailments and generally without dangerous side effects. For example, dronabinol (brand name Marinol®), a synthetic version of the naturally occurring CB₁ and CB₂ agonist tetrahydrocannabinol (discussed further below), is approved for the treatment of a number of maladies including nausea, vomiting, anorexia, and some types of pain. However, while the side effects of dronabinol are usually not dangerous, those side effects can interfere with patient quality of life. For example, the U.S. National Institutes of Health list stomach pain, confusion, sleepiness, weakness, unsteady walking, hallucinations, and memory loss as possible side effects of dronabinol, any of which could prevent a patient from successfully carrying out ordinary household and employment-related tasks. In some patients, dronabinol can cause serious side effects such as seizures or a pounding heartbeat. Researchers have as yet failed to discover an alternative medication that provides the therapeutic benefits of dronabinol but with fewer or less severe side effects.

As mentioned above, the substances whose structures are shown in FIGS. 1A-1F, as well as analogs and homologs of those substances, are used and being investigated for use in the treatment of a variety of medical problems. FIG. 1A shows the structure of arachidonoyl ethanolamide (anandamide) 12, a naturally occurring endocannabinoid synthesized by the body. Anandamine 12 is also found in chocolate along with other substances that mimic the effects of anandamide. Anandamide 12 is both a CB₁ agonist and a CB₂ agonist, and studies have determined that anandamide plays a role in, e.g., controlling eating, regulating sleep, the generation of motivation, pain relief, and other physiological and psychological processes.

FIG. 1B shows the chemical structure of 2-arachidonoyl glycerol (2-AG) 14, another naturally occurring endocannabinoid synthesized by the body. 2-AG 14 is also found in bovine milk. Like anandamide 12, 2-AG 14 is a CB₁ and CB₂ agonist, but studies on 2-AG have identified differences between the function of 2-AG and anandamide. 2-AG 14 has demonstrated beneficial effects in the treatment of glaucoma and colitis and in reducing the spread of some types of cancer cells. FIG. 1C shows the chemical structure of 2-arachidonoyl glycerol ether (2-AGE) 16. 2-AGE 16 is another naturally occurring endocannabinoid and has effects similar to those of 2-AG 14. 2-AGE 16 and 2-AG 14 are biochemically similar, but have differences in function and effect which are being investigated.

FIGS. 1D-1F show the chemical structure of cannabinoids 10 that have not been found to occur naturally in the body but which, like the endocannabinoids, act as CB₁ and/or CB₂ agonists or antagonists. Many substances shown in FIGS. 1D-1F are available in synthetic forms, and the substances shown, other cannabinoids, and analogs thereof are also found in some plants of the class Magnoliopsida, such as plants in the Asteraceae (sunflower) and Cannabaceae (hemp) families.

FIG. 1D shows the chemical structure of the cannabinoid tetrahydrocannabinol (THC) 18, the active ingredient in the prescription medication Marinol® (generic name dronabinol). Marinol is available in soft gelatin capsules containing 2.5, 5, or 10 milligrams of THC 18 along with sesame oil, gelatin, and other inactive ingredients. THC 18 is also found in some plants of the Cannabaceae (hemp) family, and in particular is present in substantial amounts in some species of the Cannabis genus. THC 18 is an agonist of both the CB₁ and CB₂ receptors, and is an approved treatment for a variety of ailments including nausea, vomiting, and lack of appetite. THC has also been found to be effective in the treatment of multiple sclerosis, glaucoma, and pain. THC 18 is prescribed in doses ranging from 2.5 to 40 milligrams per day, and patients in clinical studies have received up to 210 milligrams per day without toxic effects. THC 18 may be obtained by chemical synthesis, see, e.g., U.S. Pat. Nos. 5,292,899 and 7,186,850, herein incorporated by reference, or by extraction from THC-bearing plants using solvents such as ethanol, methanol, isopropanol, or other appropriate solvents.

FIG. 1E shows the chemical structure of the cannabinoid cannabinol (CBN) 20. CBN 20 is a byproduct of THC 18 degradation and has a weaker agonist effect on the CB₁ and CB₂ receptors than THC. Accordingly, CBN 20 has not been the subject of as much research as THC 18.

FIG. 1F shows the chemical structure of the cannabinoid cannabidiol (CBD) 22. CBD 22 is present in the prescription medication nabiximols (brand name Sativex®), which is derived from cannabis plants and contains both CBD 22 and THC 18. CBD 22 acts as a CB₁ and CB₂ antagonist and as an agonist to other non-cannabinoid receptors. Nabiximols is approved for the treatment of pain, spasticity, overactive bladder and other maladies. CBD 22 has also been shown to inhibit the growth of cancer cells and to be useful in the treatment of schizophrenia and dystonia. CBD 22 may be obtained by chemical synthesis as described by Baek et al., Tetrahedron Letters 26, 1083-1086 (1985), herein incorporated by reference, or by extraction from CBD-bearing plants using a solvent such as ethanol, methanol, isopropanol, or other appropriate solvents.

FIGS. 1A-1F show only some of the cannabinoids 10 that are used or being investigated for use as therapeutic medications, which include analogs and homologs of the substances shown and of other endocannabinoids such as docosatetraenoyl ethanolamide, N-arachidonoyl dopamine (NADA), and oleamide. Furthermore, some of the therapeutic effects of cannabinoids 10 may be caused by metabolites of the cannabinoids, i.e., by other compounds produced by reaction of the cannabinoids with other compounds within the body. Accordingly, even though the mechanism by which cannabinoids 10 produce therapeutic effects are still being investigated, cannabinoids 10 encompass an important and growing class of medications. However, the side effects of cannabinoids present a substantial obstacle to therapeutic cannabinoid use. Therefore, a need exists to reduce or eliminate the side effects of cannabinoids and researchers have long sought to discover ways to satisfy that need.

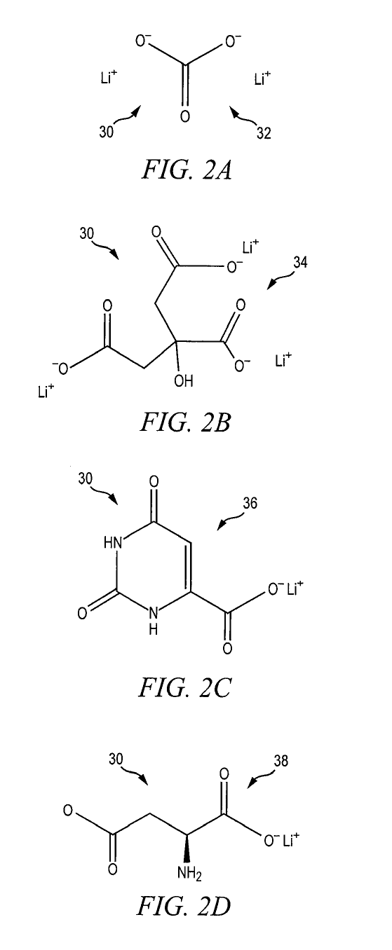

FIGS. 2A-2D show the chemical structure of some pharmacologically important lithium compounds 30. Lithium (Li) is a naturally occurring chemical element, the lightest of the metals. Lithium is highly reactive, and therefore is rarely found in pure form but instead is found in compounds. Lithium compounds are usually salts, meaning the lithium atom or atoms are bound to the other atoms in the compounds by an ionic bond. In a typical ionic bond, one atom (usually lithium in a lithium compound) gives up an electron, thereby becoming a positively charged ion, and another atom acquires an electron, thereby becoming a negatively charged ion. Because a positively charged object and a negatively charged object are attracted to each other by electrostatic force, the positively- and negatively-charge ions are bonded to each other until a stronger force breaks them apart. Lithium salts are usually water-soluble. When a lithium salt is dissolved in water, the charged ions in the salt are attracted to the surrounding water molecules and pulled apart, separating the lithium ion from the rest of the salt. Accordingly, lithium ions are found in seawater at concentrations of approximately 170 micrograms per liter. Lithium ions are also found in some mineral spring waters at concentrations of up to 500 micrograms per liter.

Lithium ions have found pharmacological use primarily in the treatment of bipolar disorder. However, the dosages of lithium used to treat bipolar disorder are very close to the toxic level, and therefore the blood levels of lithium in patients undergoing lithium treatment for bipolar disorder must be constantly monitored. At the dosages used for treating bipolar disorder, lithium has a number of side effects, including restlessness, loss of appetite, indigestion, dry mouth, hair loss, constipation, and muscle pain. Some patients experience serious side effects such as uncontrollable shaking or movement, seizures, loss of coordination, irregular heartbeat, birth defects, and hallucinations.

The mechanisms by which lithium ions act to treat bipolar disorder are not fully understood. Proposed mechanisms include, e.g., interaction with substances that transmit messages in the nervous system, deactivation of an enzyme involved in regulating sleeping and waking, promotion of neuron growth, and interaction with chemical signaling pathways within the nervous system. But while how lithium ions work within the body is still in dispute, researchers and doctors agree that lithium is an effective medication. A large number of FDA-approved clinical studies have proven the efficacy of lithium in the treatment of bipolar disorder, and therefore the FDA approved lithium for the treatment of bipolar disorder in 1970. Since that time, doctors have prescribed lithium to millions of patients.

Research indicates that lithium is an essential trace element, and several studies have attributed an increased risk of various physical and mental maladies to lithium deficiency. Accordingly, lithium compounds are marketed as nutritional supplements with purported neuro-protective, anti-aging, immunity boosting, and psychological benefits.

FIG. 2A shows the chemical structure of lithium carbonate 32, a prescription-only medication approved for the treatment of bipolar disorder. Lithium carbonate 32 is available as a tablet or capsule, optionally with a film or gelatin coating or in extended release form. Lithium carbonate 32 is approximately 19% lithium by weight. Unlike most lithium salts, lithium carbonate 32 has only limited solubility in water. Typical dosage of lithium carbonate 32 used to treat bipolar disorder range from 900 to 1800 milligrams per day, containing between 170 to 340 milligrams of lithium ions. Lithium carbonate 32 may be produced from a lithium-containing ore, e.g., spodumeme, by heating the ore to a temperature of approximately 1000° C., treating the ore using sulfuric acid, leaching the ore with water, filtering out impurities, treating the resultant solution with concentrated sodium carbonate, and filtering the solution a second time to obtain lithium carbonate. Lithium carbonate 32 may also be produced by processing lithium chloride obtained from, e.g., lithium containing brines, with sodium carbonate.

FIG. 2B shows the chemical structure of lithium citrate 34, another prescription-only medication approved for the treatment of bipolar disorder. Lithium citrate 34 is available in flavored syrup. Typical dosage of lithium citrate 34 syrup used to treat bipolar disorder ranges from 13 to 30 milliliters (3 to 6 teaspoons) per day, containing between 170 to 340 milligrams of lithium ions. Studies indicate that lithium enters the body more rapidly when delivered using lithium citrate 34 syrup, as compared to delivery using lithium carbonate 32 tablets, but that the two delivery methods are otherwise equivalent. Lithium citrate 34 may be produced by combining lithium carbonate 32 with citric acid.

FIG. 2C shows the chemical structure of lithium orotate 36. Lithium orotate 36 is available as a nutritional supplement in tablet or capsule form. A typical capsule or tablet containing 120 milligrams of lithium orotate 36 contains about 4.6 milligrams of lithium ions. Studies have found that 150 milligrams of lithium orotate 36 per day can be beneficial in the treatment of alcohol addiction. Lithium orotate 36 may be produced by combining lithium carbonate 32 with orotic acid.

FIG. 2D shows the chemical structure of lithium aspartate 38. Lithium aspartate 38 is available as a nutritional supplement in tablet or capsule form. A capsule or tablet containing 125 milligrams of lithium asparate 38 contains about 5 milligrams of lithium ions. Lithium aspartate 38 is generally considered equivalent to lithium orotate 36 as a method for delivery of therapeutic lithium ions. Lithium aspartate 38 may be produced by combining lithium carbonate 32 with aspartic acid.

In addition to the lithium compounds 30 shown in FIGS. 2A-2D, a large number of other lithium compounds, e.g., lithium chloride, may also be used to deliver therapeutic lithium ions to a human being. A practitioner of ordinary skill in the art would understand that a sufficiently bioavailable lithium compound 30 with sufficiently low toxicity could be substituted for the specific lithium compounds shown in FIGS. 2A-2D.

Patients using cannabinoid medications 10 prescribed for the treatment of symptoms such as pain and experiencing the undesirable side effects of the cannabinoid medications may provide relief through the administration of lithium compounds 30. For example, patients using a medication containing THC 18, e.g., Marinol, have experienced difficulty staying awake and maintaining concentration. Patients also reported a lack of motivation and a reduction in creativity while taking the medication. As a result, the patients have been unable or unwilling to perform ordinary tasks, such as, e.g., tasks associated with the patient's employment. However, upon treatment with lithium compounds 30, the alertness, concentration, motivation, and creativity of the patients were restored. As a result, the patients were able to continue to work productively while using the cannabinoid-based medication. Substantial restoration of alertness, concentration, motivation, and creativity was seen with doses of lithium compounds 30 containing 4 milligrams of lithium ions.

The ability of lithium compounds to restore alertness, concentration, motivation, and other abilities lost as a side effect of the administration of psychoactive substances, such as cannabinoid-based medications, is an unexpected effect of low-dose lithium therapy. For example, by using a therapeutically effective dose of lithium ions administered as one or more lithium compounds in combination with one or more cannabinoid medications, the beneficial effects of the cannabinoid medications are obtained without the sleepiness, confusion, and other undesirable side effects commonly experienced by patients using cannabinoid medications. Reducing the undesirable side effects of psychoactive medications allows patients to be more productive and enjoy a higher quality of life while under treatment with the medications. The administration to a patient of a combination of one or more lithium compounds with one or more psychoactive substances such as, for example, Marinol, can also produce improvements in the length, quality, or frequency of occurrence of sleep or relaxation experienced by the patient.

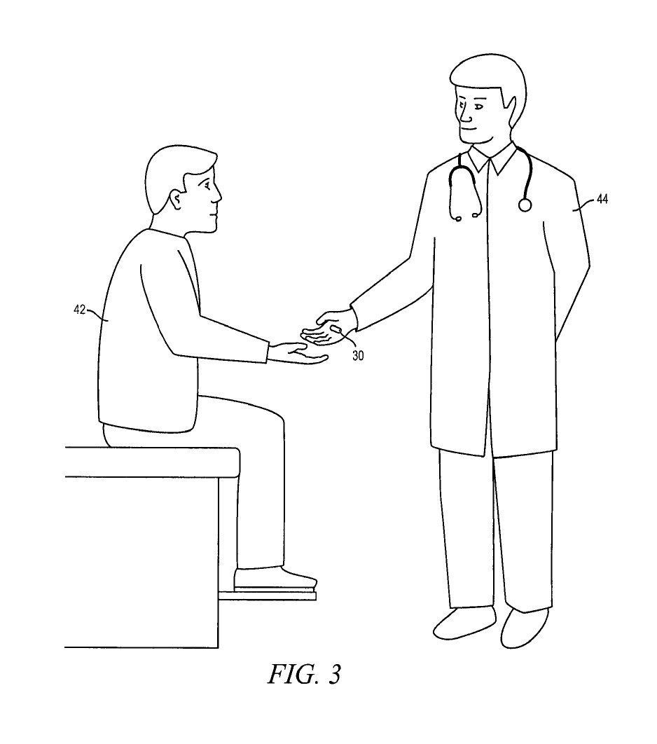

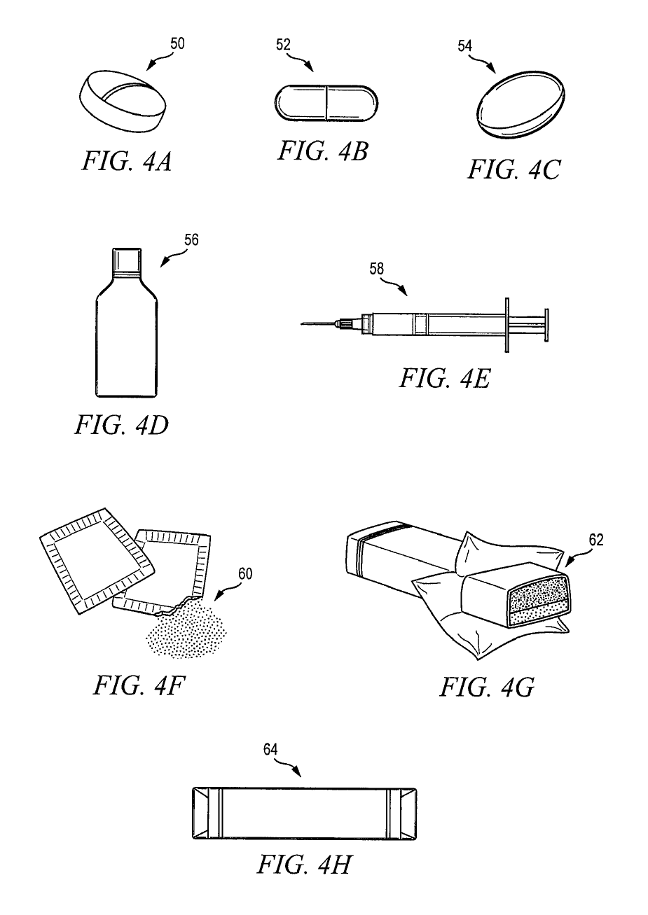

FIG. 3 shows a method of treating side effects of, for example, cannabinoid therapy in human 42 through administration of lithium compounds 30 as a prescription by medical professional 44. The administration may be in various forms as described below, including tablet or pill 50 shown in FIG. 4A. Pill 50 may be administered by medical professional 44, obtained by prescription from a pharmacist, or obtained over-the-counter from a retail or online store. Pill 50 may contain 100% of lithium compounds 30, e.g., 120 milligrams of lithium orotate providing 4.6 milligrams of lithium ion. In another embodiment, pill 50 may contain acceptable pharmaceutical carriers in some percentage, with the remaining percentage being lithium compounds 30, e.g., 75 milligrams of an acceptable pharmaceutical carrier and 52 milligrams of lithium carbonate 32 providing 10 milligrams of lithium ion. The proportions may be adjusted depending on the intended use of lithium compound 30. Alternatively, medical professional 44 may treat human 42 with a medication that includes one or more cannabinoids 10 and one or more lithium compounds 30, as described below.

The therapeutically effective dose of lithium compounds 30 providing lithium ions may vary between individual patients and with the amount and frequency of cannabinoids 10 dosage. Initially, lithium compounds 30 containing 8 milligrams of lithium ions may be administered with every milligram of orally administered cannabinoids 10. The ratio of the delivered dose of lithium ions can be adjusted when cannabinoids 10 are administered by a method that produces more efficient absorption of the cannabinoid than oral administration, such as when the cannabinoid is administered as an inhaled mist, vapor, or smoke. For example, lithium compounds 30 containing 24 milligrams of lithium ions may be administered for every one milligram of inhaled cannabinoids. Higher doses of lithium compounds 30 may be required in some patients. When the dose of lithium compounds 30 administered approaches the doses used in the treatment of bipolar disorder, the monitoring protocols generally practiced with high doses of lithium compounds should be followed to prevent toxic levels of lithium from building up in the patient.

Lithium compounds 30 may be administered at the same time as cannabinoids 10. Alternatively, lithium compounds 30 may be administered in advance of administering cannabinoids 10 to allow the lithium ions to be absorbed before the cannabinoids. For example, a therapeutically effective dose of lithium compounds 30 may be administered two to four hours before administering cannabinoids 10 so that peak plasma concentration of lithium approximately coincide with the administration of the cannabinoids. Lithium compounds 30 may also be administered after the administration of cannabinoids 10 in response to the onset of cannabinoid side effects.

The method of administration of lithium compound 30 can include various delivery vehicles, such as shown in FIGS. 4A-4H. Lithium compounds 30 can be administered by mouth using pills or tablets 50 as shown in FIG. 4A, two-piece gelatin capsules 52 as shown in FIG. 4B, soft gelcaps 54 as shown in FIG. 4C, or a liquid 56 as shown in FIG. 4D. Lithium compounds 30 may also be administered in injectable form 58 as shown in FIG. 4E. In addition, lithium compounds 30 may be administered by adding them to food in either a liquid 56 or powder 60 form, as shown in FIGS. 4D and 4F. Lithium compounds 30 may also be administered through prepared foods such as snack bars 62 shown in FIG. 4G or individually wrapped sticks of chewing gum 64 shown in FIG. 4H. A practitioner of ordinary skill in the art would understand that a variety of commonly available forms similar to those shown in FIGS. 4A-4G could also be used to administer Lithium compounds 30 or a combined lithium/cannabinoid medication as discussed below.

One or more lithium compounds 30 may be combined with one or more cannabinoids 10 to prepare a medication that delivers the therapeutic effects of the cannabinoid with a reduction in unwanted side effects. A combined lithium/cannabinoid medication may be created in any of the forms shown in FIGS. 4A-4H, among others. Because cannabinoids 10 are usually soluble in lipids such as oils and insoluble in water, and lithium compounds 30 are generally water-soluble, a combined lithium/cannabinoid medication could also include an emulsifier. For example, a combined lithium/cannabinoid medication could be provided as a liquid gelcaps 54 including 2.5 milligrams of dronabinol (THC 18), 1.5 milliliters of lithium citrate 34 syrup containing 20 milligrams of lithium ions, sesame oil, and soy lecithin or other edible emulsifier along with other substances. Alternatively, gelcaps 54 include 2.5 milligrams of dronabinol (THC 18) and 6 milliliters of lithium citrate 34 syrup containing 80 milligrams of lithium ions.

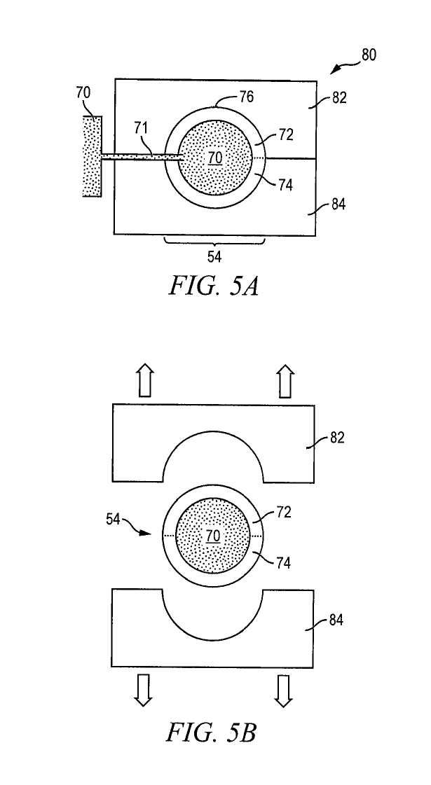

FIGS. 5A and 5B show a method of making gelcaps 54 containing lithium compounds 30 or lithium compounds 30 combined with cannabinoids 10. Fill material 70 is provided comprising lithium compounds 30 and optional cannabinoids 10 with appropriate carriers and emulsifiers. As shown in FIG. 5A, fill material 70 is injected through needle 71 between two flat ribbons 72 and 74 inside cavity 76 of die 80 comprising upper 82 and lower 84 die portion. Ribbons 72 and 74 comprise a suitable material such as gelatin or a starch- or carrageenan-based polymer. Needle 71 is withdrawn and heat is applied to seal ribbons 72 and 74 around fill material 70 to form gelcap 54 in the shape of the cavity 76. Die portions 82 and 84 are separated, as shown in FIG. 5B, and gelcaps 54 are removed from the cavity and dried.

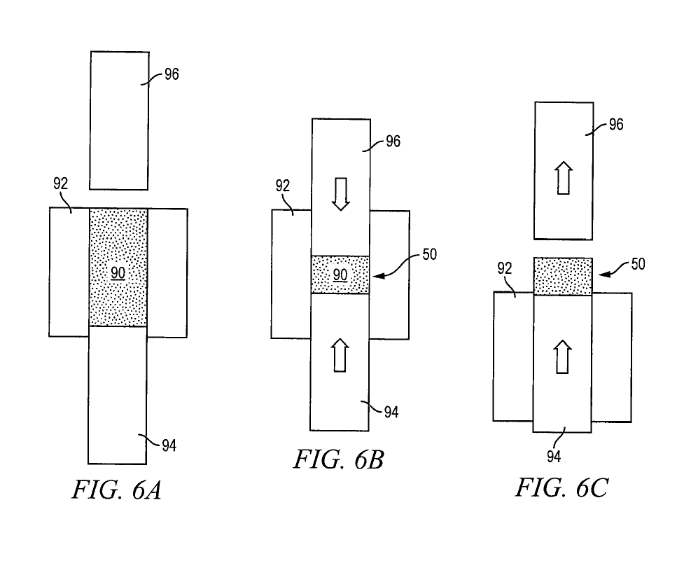

FIGS. 6A-6B show a method of making pills 50 containing lithium compounds 30 or lithium compounds combined with cannabinoids 10. Powder 90 is provided comprising lithium compounds 30 and optional cannabinoids 10 combined with suitable binders. Powder 90 is disposed within die 92 on top of lower punch 94 and beneath upper punch 96, as shown in FIG. 6A. Punches 94 and 96 are pressed together to fuse powder 90 into pill 50, as shown in FIG. 6B. Punches 94 and 96 are then moved apart and upward relative to die 92 to permit removal of pill 50 from die 92, as shown in FIG. 6C. A coating may be applied to pill 50 after molding to protect the medication and/or make the pill easier to swallow.

A practitioner of ordinary skill in the pharmaceutical packaging arts would understand that many different methods of manufacturing pharmaceutical compositions for the delivery of medications could be adapted to deliver lithium compounds 30 or lithium compounds combined with cannabinoids 10. Generally, either synthetic cannabinoids 10 or extracts containing naturally occurring cannabinoids can be used.

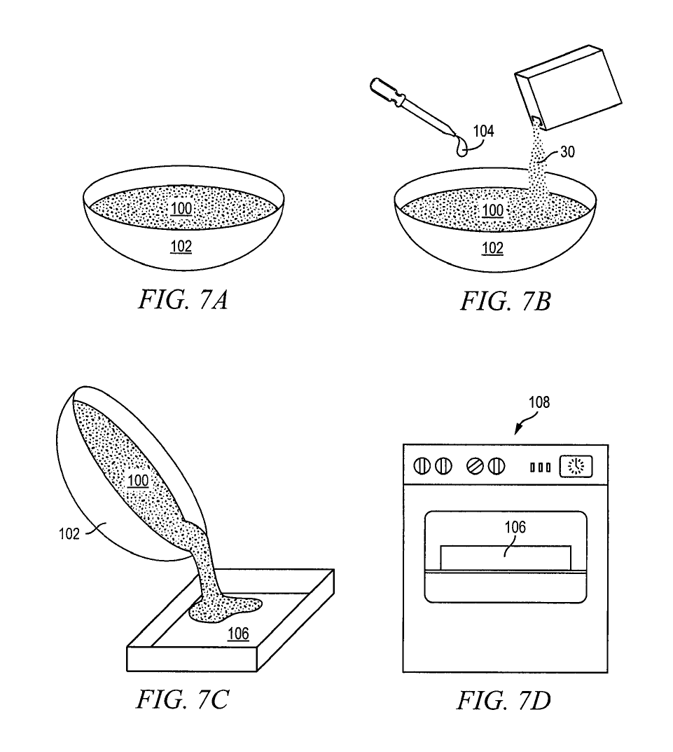

Lithium compounds 30 and cannabinoids 10 may also be provided separately or together in prepared foods such as snack bars, baked goods, confections, and candies. Providing lithium compounds 30 and cannabinoids 10 together in prepared foods may be especially advantageous for patients suffering from anorexia or nausea. FIGS. 7A-7D show the preparation of food containing lithium compounds 30 and optionally cannabinoids 10. FIG. 7A shows providing a batter or dough 100 prepared according to a conventional recipe in mixing bowl 102. FIG. 7B shows incorporating lithium compounds 30 and optional liquid containing cannabinoids 104 into batter 100. In one embodiment, a ground or powdered material containing cannabinoids is also added to batter 100. FIG. 7C shows pouring batter 100 into baking pan 106. FIG. 7D shows baking pan 106 containing batter 100 including lithium compounds 30 and optional cannabinoids 10 placed in 300° F. oven 108 for an appropriate time interval. Baking pan 106 is then removed from oven 108 and allowed to cool, after which the food may be cut into portions and served or wrapped. In one embodiment, the recipe is modified by replacing a portion of a recipe ingredient with lithium compounds 30, such as by replacing a portion of salt (sodium chloride) in the recipe with lithium chloride. The recipe may also be modified by replacing a portion of recipe ingredient with cannabinoids 10 or cannabinoid-bearing substances, such as by replacing a portion of canola or corn oil in the recipe with sesame oil containing a synthetic cannabinoid or with an oil-based extract containing naturally occurring cannabinoid. A practitioner of ordinary skill in the pertinent arts would understand that many different recipes and process for preparing prepared foods could be adapted to incorporate lithium compounds 30 or to incorporate lithium compounds and cannabinoids 10.

While one or more embodiments of the present invention have been illustrated in detail, the skilled artisan will appreciate that modifications and adaptations to those embodiments may be made without departing from the scope of the present invention as set forth in the following claims.

1. A method of mitigating side effects of a psychoactive substance, comprising administering a therapeutically effective dose of lithium ions.

2. The method of claim 1, wherein the therapeutically effective dose of lithium ions includes greater than 4 milligrams of lithium and less than 170 milligrams of lithium.

3. The method of claim 1, wherein the therapeutically effective dose of lithium ions includes between 8 and 32 milligrams of lithium ions per milligram of the psychoactive substance.

4. The method of claim 1, further including administering the therapeutically effective dose of lithium ions using lithium carbonate, lithium citrate, lithium chloride, lithium orotate, lithium aspartate, or analogs thereof.

5. The method of claim 1, further including administering the therapeutically effective dose of lithium ions prior to administration of the psychoactive substance.

6. The method of claim 1, further including administering the therapeutically effective dose of lithium ions in response to the side effects of the psychoactive substance.

7. The method of claim 1, further including administering the therapeutically effective dose of lithium ions simultaneously with the psychoactive substance.

8. The method of claim 1, further including administering the therapeutically effective dose of lithium ions using a delivery vehicle selected from pills, tablets, capsules, gelcaps, liquids, syrups, injectable liquids, powders, or foods.

9. The method of claim 8, further including administering the psychoactive substance using the delivery vehicle.

10. The method of claim 1, wherein the psychoactive substance includes one or more cannabinoids or analogs thereof.

11. The method of claim 1, wherein mitigating side effects of the psychoactive substance includes improving a length, quality, or frequency of occurrence of sleep or relaxation.

12. A pharmaceutical composition for mitigating side effects of a psychoactive substance, comprising a therapeutically effective dose of lithium ions.

13. The pharmaceutical composition of claim 12, wherein the pharmaceutical composition includes a pill, tablet, capsule, gelcap, liquid, syrup, injectable liquid, powder, or food.

14. The pharmaceutical composition of claim 12, wherein the therapeutically effective dose of lithium ions is provided using lithium carbonate, lithium citrate, lithium chloride, lithium orotate, lithium aspartate, or analogs thereof.

15. The pharmaceutical composition of claim 12, wherein the therapeutically effective dose of lithium ions includes greater than 4 milligrams and less than 170 milligrams of lithium ions.

16. The pharmaceutical composition of claim 12, further including a therapeutically effective dose of the psychoactive substance.

17. The pharmaceutical composition of claim 12, wherein the psychoactive substance includes one or more of anandamide, 2-arachidonoyl glycerol, 2-arachidonoyl glycerol ether, tetrahydrocannabinol, cannabinol, cannabidiol, or analogs thereof.

18. A method of making a pharmaceutical composition for mitigating side effects of a psychoactive substance, comprising:
providing a lithium compound including a therapeutically effective dose of lithium ions;
providing the psychoactive substance; and
incorporating the lithium compound and the psychoactive substance into a delivery vehicle.

19. The method of claim 18, wherein the delivery vehicle includes a pill, tablet, capsule, gelcap, liquid, syrup, injectable liquid, powder, or food.

20. The method of claim 18, wherein incorporating the lithium compound and the psychoactive substance into the delivery vehicle includes compressing the lithium compound and the psychoactive substance inside a die.

21. The method of claim 18, wherein incorporating the lithium compound and the psychoactive substance into the delivery vehicle includes disposing the lithium compound and the psychoactive substance within a capsule comprising gelatin, a starch-based polymer, or a carrageenan-based polymer.

22. The method of claim 18, wherein the psychoactive substance includes a cannabinoid.

23. A method of making a pharmaceutical composition for mitigating side effects of a psychoactive substance, comprising:
providing a lithium compound including a therapeutically effective dose of lithium ions; and
incorporating the lithium compound into a delivery vehicle.

24. The method of claim 23, wherein the delivery vehicle includes a pill, tablet, capsule, gelcap, liquid, syrup, injectable liquid, powder, or food.

25. The method of claim 23, wherein incorporating the lithium compound into the delivery vehicle includes compressing the lithium compound inside a die.

26. The method of claim 23, wherein incorporating the lithium compound into the delivery vehicle includes disposing the lithium compound within a capsule comprising gelatin, a starch-based polymer, or a carrageenan-based polymer.

27. The method of claim 23, wherein incorporating the lithium compound into the delivery vehicle includes:
preparing a batter or dough;
incorporating the lithium compound into the batter or dough; and
applying heat to the batter or dough.
